(19) United States
(12) Patent Application Publication
(10) Pub. No.: US 2024/0418689 A1
(43) Pub. Date: Dec. 19, 2024

(54) Detection Method of Polyamines, and Diagnostic Method of Cancers Using the Same

(71) Applicant: KAOHSIUNG MEDICAL UNIVERSITY, Kaohsiung (TW)

(72) Inventors: Chia-Hsien Feng, Kaohsiung (TW); Wen-Rong Chen, Kaohsiung (TW); Chi-Yu Lu, Kaohsiung (TW)

(21) Appl. No.: 18/735,231

(22) Filed: Jun. 6, 2024

Related U.S. Application Data
(60) Provisional application No. 63/472,670, filed on Jun. 13, 2023.

(30) Foreign Application Priority Data
May 27, 2024 (TW) 113119537

(51) Int. Cl. G01N 31/22; G01N 21/75; G01N 33/50

(52) U.S. Cl. CPC G01N 31/22; G01N 21/75; G01N 33/50

(57) ABSTRACT

A detection method of polyamines is used to solve the problem that the conventional method is not suitable for detecting polyamines. The detection method of polyamines includes providing a sample with polyamines. The sample and a derivatization reagent with an isothiocyanate group are dissolved in a working solution to form a mixture, while a thiocarbamoylation reaction between the polyamine in the sample and the derivatization reagent is performed to obtain a derivatization solution with a thiourea derivative. The thiourea derivative in the derivatization solution is then detected to obtain a value of polyamine. With such performance, polyamine in the sample can be effectively detected. A diagnostic method of cancers is also disclosed.

### CROSS REFERENCE TO RELATED APPLICATIONS

The application claims the benefit of U.S. provisional application No. 63/472,670, filed on Jun. 13, 2023, and the benefit of Taiwan application serial No. 113119537, filed on May 27, 2024, the subject matter of which are incorporated herein by reference.

### BACKGROUND OF THE INVENTION

### 1. Field of the Invention

The present invention generally relates to a detection method and, more particularly, to a detection method of polyamines. The present invention also relates to a diagnostic method of cancers using the detection method.

### 2. Description of the Related Art

Polyamines are organic compounds with two or more amino groups (—NH₂). The most common examples include spermidine (classified as a triamine), spermine (classified as a tetraamine) and putrescine (a precursor diamine of spermidine and spermine; classified as a diamine).

Polyamines can be protonated to form cationic molecules, binding to nucleic acids and chromatins, and modulating signal transduction pathways. When intracellular biosynthesis of polyamines is inhibited, cell growth will be severely hindered or stopped. Moreover, in vigorously growing cancer tissues, the biosynthesis and secretion of polyamines will increase significantly, so the content of polyamines is considered to be cancer biomarkers.

In addition, it is known that long-term intake of high levels of polyamines will also increase the levels of polyamines in the body, thereby increasing the risk of cancer. There are also methods of reducing the intake of dietary polyamines to treat cancer [named polyamine blocking therapy (PBT)]. Therefore, if the polyamines content in food can be detected, it can also provide a reference for people's dietary intake.

However, since most polyamines are highly polar substances, do not have specific UV luminophores, and lack fluorescent properties, polyamines are difficult to detect using conventional detection methods. Accordingly, a detection method of polyamines is still needed.

### SUMMARY OF THE INVENTION

It is therefore an objective of the present invention to provide a detection method of polyamines to effectively detect polyamines in a sample.

It is another objective of the present invention to provide a detection method of polyamines with small amount of sample.

It is yet another objective of this invention to provide a diagnostic method of cancers using the detection method of polyamines.

As used herein, the term “a”, “an” or “one” for describing the number of the elements and members of the present invention is used for convenience, providing the general meaning of the scope of the present invention, and should be interpreted to include one or at least one. Furthermore, unless explicitly indicated otherwise, the concept of a single component also includes the case of plural components.

One embodiment of the present invention discloses a detection method of polyamines. The detection method can comprise: providing a sample with polyamines. The sample and a derivatization reagent with an isothiocyanate group are dissolved in a working solution to form a mixture. A thiocarbamoylation reaction between the polyamines in the sample and the isothiocyanate group of the derivatization reagent is performed to obtain a derivatization solution with a thiourea derivative. The thiourea derivative is detected to obtain a value of polyamines.

Accordingly, by the thiocarbamoylation reaction between the amino group of the polyamines in the sample and the derivatization reagent (an isothiocyanate with the isothiocyanate group), the thiourea derivative can be obtained. The thiourea derivative can be detected by various conventional methods. For example, after separation by liquid chromatography, gas chromatography, etc., the thiourea derivative can be detected using ultraviolet spectroscopy, fluorescence spectroscopy, or mass spectrometry. As such, the detection method of polyamines according to the present invention has great sensitivity, precision and accuracy, reducing sample usage.

In the detection method of polyamines, the derivatization reagent can be 4-dimethylamino-naphthylisothiocyanate (DNITC), 1-naphthyl isothiocyanate (NITC), benzyl isothiocyanate (BITC) or ally isothiocyanate (AITC). As such, by the use of specific derivatization reagent, the amount of thiourea derivative formed by the thiocarbamoylation reaction between the polyamines and the isothiocyanate group of the derivatization reagent can be increased, further improving the detection effect of polyamines.

In the detection method of polyamines, the working solution can be acetonitrile (ACN), acetone, dimethyl sulfoxide (DMSO), pyridine, dimethylformamide (DMF), dimethylpropyleneurea (DMPU), sulfolane, tetrahydrofuran (THF), 1,2-dimethoxyethane (DME), 1,3-dioxolane (DOL), dimethyl carbonate (DMC) or water. As such, by the use of specific working solution, the solubility of the sample and the derivatization reagent can be increased, further improving the detection effect of polyamines.

The detection method of polyamines can further comprise: microwaving the mixture to drive the performance of the thiocarbamoylation reaction. For example, the mixture can be microwaved at a power of from 100 watts to 750 watts. As such, by the energy provided by microwave the thiocarbamoylation reaction can be accelerated, further improving the detection efficiency of further improving the detection efficiency of polyamines.

The detection method of polyamines can further comprise: adding a base to the mixture, followed by performing the thiocarbamoylation reaction in the mixture dissolved with the base. For example, the base can be sodium hydroxide (NaOH), sodium bicarbonate (NaHCO₃), sodium carbonate (Na₂CO₃), 4-dimethylaminopyridine (DMAP) or triethylamine (TEA). As such, by the addition of the base, the amino group of the polyamines can be deprotonated to increase the nucleophilicity of the amino group, accelerating the thiocarbamoylation reaction, further improving the detection efficiency of polyamines.

The detection method of polyamines can further comprise: adding a removing reagent to the derivatization solution to remove fat-soluble interferents in the derivatization solution by the removing reagent and to form an upper-layer solution and a lower-layer solution. The lower-layer solution contains the thiourea derivative. The removing reagent is a solvent that is immiscible with the derivation solution and has a log P of from 3.5 to 7.0. For example, the removing reagent can be hexane, octane, decane or dodecane. As such, by the addition of the removing reagent, excessive derivatization reagent can be effectively removed, preventing the excessive derivatization reagent from affecting subsequent analysis results. Accordingly, the detection accuracy of polyamines can be improved.

The detection method of polyamines can further comprise: adding a proton donor and an effervescent salt to the derivatization solution to produce carbon dioxide (CO_{2(g)}) by a neutralization reaction between the proton donor and the effervescent salt, and to form an upper-layer solution and a lower-layer solution. The upper-layer solution contains the thiourea derivative. For example, the proton donor can be citric acid, ammonium chloride (NH₄Cl), ammonium sulfate ((NH₄)₂SO₄) or sodium dihydrogen phosphate (NaH₂PO₄), and the effervescent salt can be potassium bicarbonate (KHCO₃), sodium bicarbonate (NaHCO₃), ammonium bicarbonate (NH₄HCO₃), potassium carbonate (K₂CO₃) or sodium carbonate (Na₂CO₃). As such, by the addition of the proton donor and effervescent salt, carbon dioxide (CO_{2(g)}) can be produced to promote the dispersion of molecules in the derivatization solution, further improving the detection efficiency of polyamines.

The detection method of polyamines can further comprise: after adding the proton donor and the effervescent salt to the derivatization solution, and before forming the upper-layer solution and the lower-layer solution, the derivatization solution is placed in a cooling bath. As such, by the use of the cooling bath, the upper-layer solution and the lower-layer solution can be effectively layered, and thereby the thiourea derivative can be effectively separated. Accordingly, the detection accuracy of polyamines can be improved.

The detection method of polyamines can further comprise: adding a removing reagent, a proton donor and an effervescent salt to the derivatization solution to remove fat-soluble interferents in the derivatization solution by the removing reagent, to produce carbon dioxide (CO_{2(g)}) by a neutralization reaction between the proton donor and the effervescent salt, and to form a upper-layer solution, a middle-layer solution and a lower-layer solution. The middle-layer solution contains the thiourea derivative. The removing reagent is a solvent that is immiscible with the derivation solution and has a log P of from 3.5 to 7.0. For example, the removing reagent can be hexane, octane, decane or dodecane, the proton donor can be citric acid, ammonium chloride (NH₄Cl), ammonium sulfate ((NH₄)₂SO₄) or sodium dihydrogen phosphate (NaH₂PO₄), and the effervescent salt can be potassium bicarbonate (KHCO₃), sodium bicarbonate (NaHCO₃), ammonium bicarbonate (NH₄HCO₃), potassium carbonate (K₂CO₃) or sodium carbonate (Na₂CO₃). As such, by the addition of the removing reagent, excessive derivatization reagent can be effectively removed, preventing the excessive derivatization reagent from affecting subsequent analysis results. Accordingly, the detection accuracy of polyamines can be improved. Moreover, by the addition of the proton donor and effervescent salt, carbon dioxide (CO_{2(g)}) can be produced to promote the dispersion of molecules in the derivatization solution, further improving the detection efficiency of polyamines.

The detection method of polyamines can further comprise: after adding the removing reagent, the proton donor and the effervescent salt to the derivatization solution, and before forming the upper-layer solution, the middle-layer solution and the lower-layer solution, the derivatization solution is placed in a cooling bath. As such, by the use of the cooling bath, the upper-layer solution, the middle-layer solution and the lower-layer solution can be effectively layered, and thereby the thiourea derivative can be effectively separated. Accordingly, the detection accuracy of polyamines can be improved.

Another embodiment of the present invention discloses a diagnostic method of cancers. The diagnostic method can comprise: obtaining a biological sample from a suspected patient. Polyamines in the biological sample is detected ex vivo by the detection method mentioned above to obtain a detection value. The detection value of the biological sample is compared with a reference value, while the detection value is higher than the reference value indicates that the suspected patient suffers from cancer. For example, the cancer can be liver cancer, prostate cancer, pancreatic cancer, colorectal cancer, breast cancer, lymphoma, fibroma, lung cancer, gastric cancer or oral cancer.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 depicts a flow chart of a detection method of polyamines according to a first embodiment of the present invention.

FIG. 2 depicts a chemical equation of a thiocarbamoylation reaction between a polyamine and a derivatization reagent (isothiocyanate).

FIG. 3 depicts a chemical structure of a derivatization reagent, 4-dimethylamino-naphthylisothiocyanate (DNITC).

FIG. 4 depicts a chemical structure of a derivatization reagent, 1-naphthyl isothiocyanate (NITC).

FIG. 5 depicts a chemical structure of a derivatization reagent, benzyl isothiocyanate (BITC).

FIG. 6 depicts a chemical structure of a derivatization reagent, ally isothiocyanate (AITC).

FIG. 7 depicts a flow chart of a detection method of polyamines according to a second embodiment of the present invention.

FIG. 8 depicts a flow chart of a detection method of polyamines according to a third embodiment of the present invention.

FIG. 9 depicts a flow chart of a detection method of polyamines according to a fourth embodiment of the present invention.

FIG. 10 depicts a chemical structure of N,N-dimethyl-1-naphthylamine (DNA).

FIG. 11 depicts a chemical structure of naphthalene.

FIG. 12 depicts a bar chart illustrating relative response (value of polyamines) of the thiourea derivative in the derivatization solutions of groups A1 to A6 obtained by the detection method of polyamines according to the fourth embodiment of the present invention in trial (A).

FIG. 13 depicts a bar chart illustrating relative response (value of polyamines) of the thiourea derivative in the derivatization solutions of groups B0 to B5 obtained by the detection method of polyamines according to the fourth embodiment of the present invention in trial (B).

FIG. 14 depicts a bar chart illustrating relative response (value of polyamines) of the thiourea derivative in the derivatization solutions of groups C0 to C4 obtained by the detection method of polyamines according to the fourth embodiment of the present invention in trial (C).

FIG. 15 depicts a bar chart illustrating relative response (value of polyamines) of the thiourea derivative in the derivatization solutions of groups D0 to D4 obtained by the detection method of polyamines according to the fourth embodiment of the present invention in trial (D).

FIG. 16 depicts a bar chart illustrating relative response (value of polyamines) of the thiourea derivative in the derivatization solutions of groups E0 to E3 obtained by the detection method of polyamines according to the fourth embodiment of the present invention in trial (E).

FIG. 17 depicts a bar chart illustrating relative response (value of polyamines) of the thiourea derivative in the derivatization solutions of groups F1 to F4 obtained by the detection method of polyamines according to the fourth embodiment of the present invention in trial (F).

FIG. 18 depicts a bar chart illustrating relative response (value of polyamines) of the thiourea derivative in the derivatization solutions of groups G1 to G5 obtained by the detection method of polyamines according to the fourth embodiment of the present invention in trial (G).

FIG. 19 depicts a bar chart illustrating relative response (value of polyamines) of the thiourea derivative in the derivatization solutions of groups H0 to H1 obtained by the detection method of polyamines according to the fourth embodiment of the present invention in trial (H).

FIG. 20 depicts chromatograms of the derivatization solutions of groups I1 to I3 obtained by the detection method of polyamines according to the fourth embodiment of the present invention in trial (I). Peaks 1, 2 and 3 respectively indicate the thiourea derivatives of putrescine, spermidine and spermine, while peak DNITC indicates the peak of derivatization reagent (DNITC).

FIG. 21 depicts a line chart illustrating relative response (value of polyamines) of the thiourea derivative in the derivatization solution obtained by the detection method of polyamines according to the fourth embodiment of the present invention in trial (J).

### DETAILED DESCRIPTION OF THE INVENTION

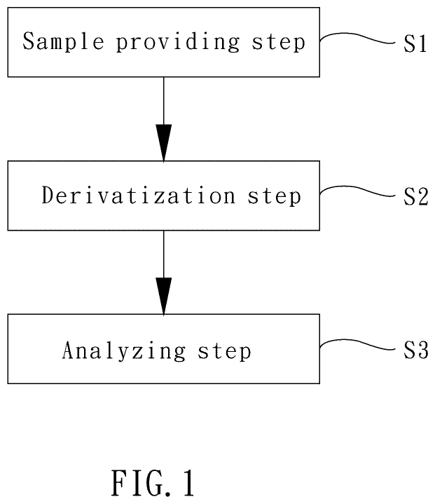

Referring to FIG. 1, a detection method of polyamines according to a first embodiment of the present invention can include: a sample providing step S1, a derivatization step S2 and an analyzing step S3.

Specifically, in the sample providing step S1, a sample with polyamines is provided. For example, the sample can be a food sample or a biological sample [such as a blood sample, a urine sample, a salivary sample, a cerebrospinal fluid (CSF) sample or an oral tissue sample]. If the matrix of the sample is too complex, such as containing macromolecules like protein, the sample can be first mixed with a protein precipitant. The protein precipitant can be sodium hydroxide (NaOH), acetonitrile (ACN), methanol, ethanol, hydrochloric acid (HCl), ammonium sulfate ((NH₄)₂SO₄), etc. The protein in the sample can thus form a protein precipitate, and the protein precipitate can be further removed by centrifugation (e.g., 15,000 rpm for 2 minutes), obtaining a supernatant. The supernatant can be use as the sample for subsequent derivatization step S2.

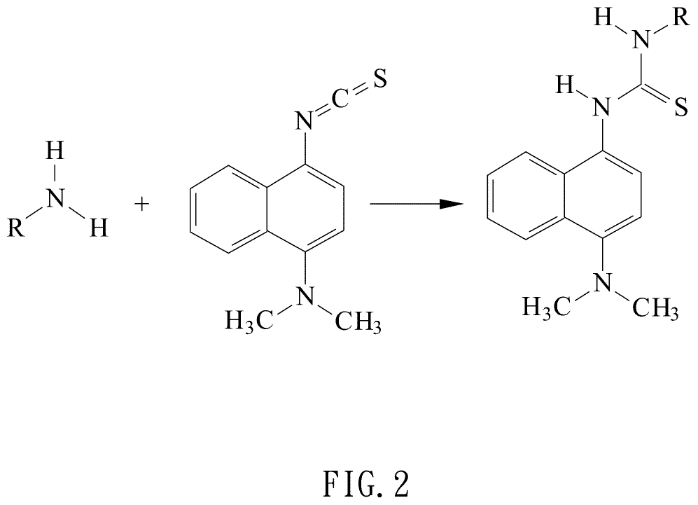

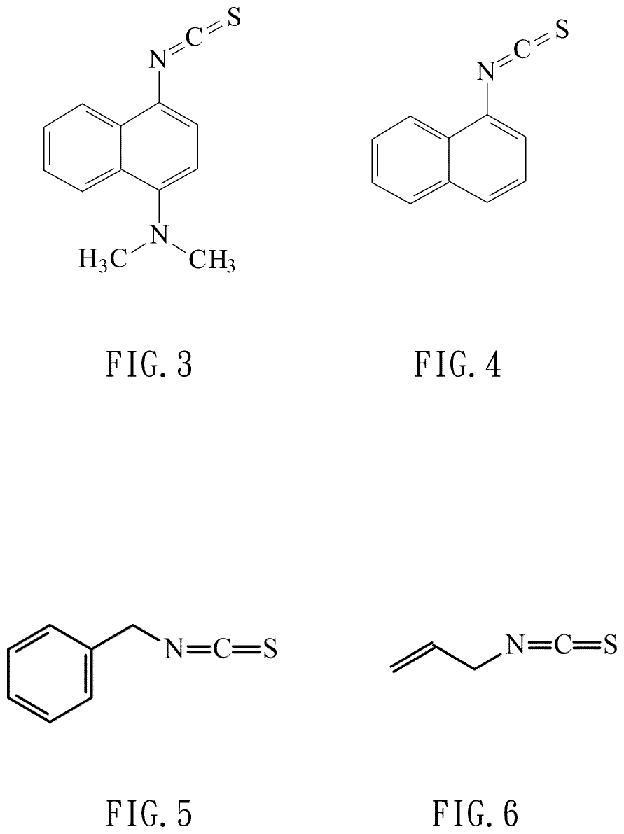

In the derivatization step S2, an isothiocyanate with an isothiocyanate group can be used as a derivatization reagent. The derivatization reagent is dissolved in a working solution, and the sample is added to form a mixture. As such, a thiocarbamoylation reaction shown in FIG. 2 between the derivatization reagent and the polyamines in the sample can be carried out to form a derivatization solution, which includes a thiourea derivative. For example, in the mixture, the derivatization reagent can be isothiocyanates such as 4-dimethylamino-naphthylisothiocyanate (DNITC, with a chemical structure shown in FIG. 3), 1-naphthyl isothiocyanate (NITC, with a chemical structure shown in FIG. 4), benzyl isothiocyanate (BITC, with a chemical structure shown in FIG. 5) or ally isothiocyanate (AITC, with a chemical structure shown in FIG. 6) with a concentration of from 5 mM to 15 mM. Moreover, the working solution is a solvent which can dissolve the derivatization reagent and the polyamines in the sample, thereby forming the mixture together with the derivatization reagent and the polyamines. For example, the working solution can be solvents such as acetonitrile (ACN), acetone, dimethyl sulfoxide (DMSO), pyridine, dimethylformamide (DMF), dimethylpropyleneurea (DMPU), sulfolane, tetrahydrofuran (THF), 1,2-dimethoxyethane (DME), 1,3-dioxolane (DOL), dimethyl carbonate (DMC) or water.

In order to accelerate the thiocarbamoylation reaction, a base can be added to the mixture. The base is a basic compound soluble in the working solution. As such, by the addition of the base, the amino group of the polyamines can be deprotonated to increase the nucleophilicity of the amino group, accelerating the thiocarbamoylation reaction. For example, in the mixture, the base can be the basic compounds such as sodium hydroxide (NaOH), sodium bicarbonate (NaHCO₃), sodium carbonate (Na₂CO₃), 4-dimethylaminopyridine (DMAP) or triethylamine (TEA) with a concentration of from 5 mM to 75 mM.

Furthermore, in order to accelerate the thiocarbamoylation reaction, the mixture can also be heated. For example, the mixture can be placed at a temperature of from 30° C. to 60° C. for a time period of from 10 minutes to 24 hours. Alternatively, the mixture can be heated by microwave. For example, the mixture can be microwaved for a time period of from 0.5 minutes to 10 minutes using a microwave oven with a power of from 100 watts to 750 watts.

Then, in the analyzing step S3, the derivatization solution can be used as an analytic solution, and the thiourea derivative in the analytic solution can be detected by means of various conventional methods, obtaining a value of polyamines. The value of polyamines can be used to evaluate whether the sample comprises polyamines and the polyamine level in the sample. For example, after separation by liquid chromatography, gas chromatography, etc., the thiourea derivative in the analytic solution can be detected using ultraviolet spectroscopy, fluorescence spectroscopy, or mass spectrometry. Specifically, the conventional methods can be high performance liquid chromatography with ultraviolet detector (HPLC-UV), high performance liquid chromatography with diode-array detector (HPLC-DAD), high performance liquid chromatography with fluorescence detector (HPLC-FLD), liquid chromatography-mass spectrometry (LC-MS), liquid chromatography-mass spectrometry/mass spectrometry (LC-MS/MS), matrix-assisted laser desorption ionization-time of flight mass spectrometry (MALDI-TOF MS), gas chromatography-mass spectrometry (GC-MS), gas chromatography-flame ionization detector (GC-FID), etc.

As an example, when the thiourea derivative is separated by liquid chromatography, a C18 column (2.1 mm×50 mm; particle size: 2.7 μm) can be used as the stationary phase. Moreover, a gradient elution is performed using a mixture shown in TABLE 1 as the mobile phase. The elution flow rate is set as 180 μL/min.

TABLE 1

mixture

| | Solution A | |
|---|---|---|
| Time | (0.15% aqueous | Solution B |
| (minutes) | formic acid solution) | (methanol) |
| 0.0→6.0 | 80%→60% | 20%→40% |
| 6.0→7.0 | 60%→40% | 40%→60% |
| 7.0→11.8 | 40%→40% | 60%→60% |
| 11.8→13.3 | 40%→0% | 60%→100% |
| 13.3→20.0 | 0%→0% | 100%→100% |

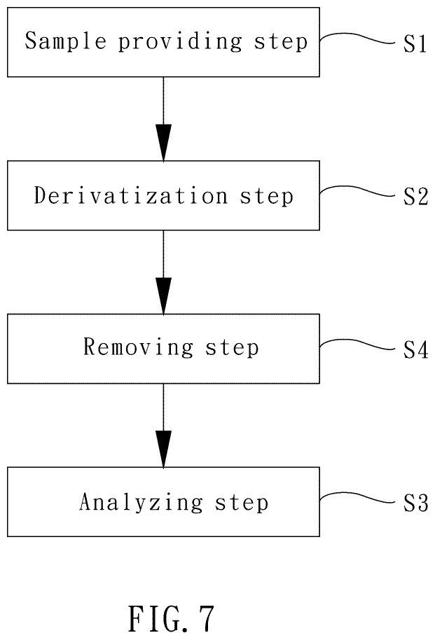

Referring to FIG. 7, since the analytic solution (the derivatization solution) may contain excessive derivatization reagent (isothiocyanate with the isothiocyanate group) during the analyzing step S3, the detection method of polyamines according to a second embodiment of the present invention can further comprise a removing step S4, which is carried out after the derivatization step S2 and before the analyzing step S3.

In the removing step S4, a removing reagent is added to the derivatization solution. The removing reagent is a solvent that is immiscible with the derivation solution and has a log P of from 3.5 to 7.0. As such, fat-soluble interferents such as the derivation reagent (the isothiocyanate with the isothiocyanate group) in the derivation solution can be removed, and the derivation solution can be also layered to form an upper-layer solution and a lower-layer solution. The lower-layer solution contains the thiourea derivative. For example, the removing reagent can be solvents such as hexane, octane, decane or dodecane. Moreover, as for the derivatization solution with a total volume of from 100 μL to 500 μL, the adding volume of the removing reagent can be of 100 μL to 1,000 μL. In addition, in the situation that the derivation solution contains a very large amount of fat-soluble interferents, the worker can also choose to execute the removing step S4 multiple times in succession. That is, after obtaining the lower-layer solution, the removing reagent can be added to the lower-layer solution. The lower-layer solution will be layered to form another upper-layer solution and another lower-layer solution. At this time, the another lower-layer solution contains the thiourea derivative.

After the removing step S4, the lower-layer solution containing the thiourea derivative can be used as the analytic solution, and the thiourea derivative in the analytic solution can be detected by the above-mentioned conventional method to obtain the value of polyamines.

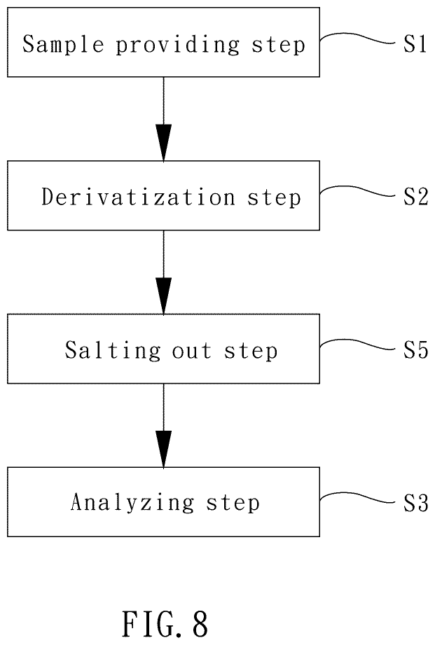

Referring to FIG. 8, in order to remove undesired anions and cations in the derivation solution, the detection method of polyamines according to a third embodiment of the present invention can further comprise a salting out step S5, which is carried out after the derivatization step S2 and before the analyzing step S3.

In the salting out step S5, a proton donor and an effervescent salt is added to the derivatization solution. The proton donor is used to provide hydrogen ions (H⁺), and the effervescent salt in the derivatization solution dissociates to form an anion and a cation, and to produce carbon dioxide (CO_{2(g)}) by a neutralization reaction with the hydrogen ions (H⁺) provided by the proton donor. After adding the proton donor and the effervescent salt to the derivatization solution, carbon dioxide (CO_{2(g)}) in form of bubbles can be produced in the derivatization solution, thereby increasing the dispersion of molecules in the derivatization solution, which is helpful to salting out. At the same time, under the action of the proton donor and the effervescent salt, the derivatization solution can be layered to form an upper-layer solution and a lower-layer solution. The upper-layer solution contains the thiourea derivative. For example, the proton donor can be citric acid, ammonium chloride (NH₄Cl), ammonium sulfate ((NH₄)₂SO₄), sodium dihydrogen phosphate (NaH₂PO₄), etc., while the effervescent salt can be potassium bicarbonate (KHCO₃), sodium bicarbonate (NaHCO₃), ammonium bicarbonate (NH₄HCO₃), potassium carbonate (K₂CO₃), sodium carbonate (Na₂CO₃), etc. Moreover, as for the derivatization solution with a total volume of from 100 μL to 500 μL, the adding weight of the proton donor (in the form of a solid) can be of 5 mg to 75 mg, while the adding weight of the effervescent salt (in the form of a solid) can be of 5 mg to 75 mg.

It is worthy to note that in the salting out step S5, after adding the proton donor and the effervescent salt to the derivatization solution, and before forming the upper-layer solution and the lower-layer solution, the derivatization solution can be placed in a cooling bath (with a temperature of −70° C. to 0° C.) to promote the effective layering of the derivatization solution.

After the salting out step S5, the upper-layer solution containing the thiourea derivative can be used as the analytic solution, and the thiourea derivative in the analytic solution can be detected by the above-mentioned conventional method to obtain the value of polyamines.

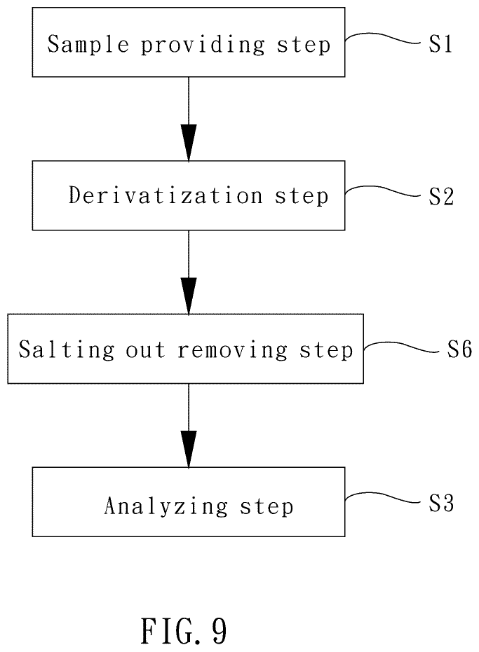

Referring to FIG. 9, in the detection method of polyamines according to a fourth embodiment of the present invention, a salting out removing step S6 is carried out after the derivatization step S2 and before the analyzing step S3.

In the salting out removing step S6, the removing reagent, the proton donor and the effervescent salt are added to the derivatization reagent at the same time. The functions, types and adding amounts of the removing reagent, the proton donor and the effervescent salt can all be as described above. Detail description is omitted to avoid redundancy. After adding the removing reagent, the proton donor and the effervescent salt to the derivatization solution, the derivatization solution can be layered to form an upper-layer solution, a middle-layer solution and a lower-layer solution. The middle-layer solution contains the thiourea derivative. Similarly, after adding the removing reagent, the proton donor and effervescent salt to the derivatization solution, and before forming the upper-layer solution, the middle-layer solution and the lower-layer solution, the derivatization solution can be placed in the cooling bath to promote the effective layering of the derivatization solution.

Then, the middle-layer solution containing the thiourea derivative can be used as the analytic solution, and the thiourea derivative in the analytic solution can be detected by the above-mentioned conventional method to obtain the value of polyamines.

According to the foregoing, based on the same technical concept, a detection kit of polyamines according to a first embodiment of the present invention can comprise: the derivatization reagent and the working solution. The derivatization reagent is the isothiocyanate with the isothiocyanate group, while the working solution is the solvent which can dissolve the derivatization reagent and the polyamines in the sample. Moreover, the roles and the specific examples of the derivatization reagent and the working solution can all be as described above. Detail description is omitted to avoid redundancy.

In addition, in order to accelerate the thiocarbamoylation reaction, the detection kit of polyamines can further comprise the base. The base is the basic compound soluble in the working solution. Again, the role and the specific examples of the base can all be as described above. Detail description is omitted to avoid redundancy.

Corresponding to the detection method of polyamines according to the second embodiment of the present invention shown in FIG. 7, a detection kit of polyamines according to a second embodiment of the present invention may also include the removing reagent in addition to the derivation reagent and the working solution. The removing reagent is the solvent with a log P of from 3.5 to 7.0. Moreover, the role and the specific examples of the removing reagent can all be as described above. Detail description is omitted to avoid redundancy.

Corresponding to the detection method of polyamines according to the third embodiment of the present invention shown in FIG. 8, a detection kit of polyamines according to a third embodiment of the present invention may also include the proton donor and the effervescent salt in addition to the derivation reagent and the working solution. The proton donor is used to provide hydrogen ions (H⁺), and the effervescent salt is used to produce carbon dioxide (CO_{2(g)}) by a neutralization reaction with the proton donor. Moreover, the roles and the specific examples of the proton donor, as well as the effervescent salt, can all be as described above. Detail description is omitted to avoid redundancy.

Corresponding to the detection method of polyamines according to the fourth embodiment of the present invention shown in FIG. 9, a detection kit of polyamines according to a fourth embodiment of the present invention may also include the removing reagent, the proton donor and the effervescent salt in addition to the derivation reagent and the working solution. The properties, roles and specific examples of the removing reagent, the proton donor and the effervescent salt can all be as described above. Detail description is omitted to avoid redundancy.

It is worthy to note that by means of the detection method of polyamines, as well as the detection kit of polyamines which is applied to carry out the detection method, can be used to detect the biological sample derived from a mammal. The detection method of polyamines, as well as the detection kit of polyamines, can further apply to detect the polyamine level in a suspected patient, thereby evaluating whether the suspected patient suffers from cancer. Specially, the biological sample is obtained from the suspected patient. Then, the polyamines in the biological sample is detected ex vivo by the detection method of polyamines to obtain a detection value. Subsequently, the detection value of the biological sample is compared with a reference value. Finally, the suspected patient is considered as a patient suffering from cancer when the detection value is higher than the reference value.

For example, Shirakawa et al. reported that the spermidine content in the red blood cells of healthy adults was 15.04±3.63 ng/g and the spermine content was 8.82±3.12 ng/g, while the spermidine content in the red blood cells of pancreatic cancer patients was 47.15±25.97 ng/g and the spermine content was 12.27±9.44 ng/g, and the spermidine in the red blood cells of colorectal cancer patients was 31.97±23.29 ng/g, and the spermine was 41.59±37.57 ng/g, indicating the detection value of polyamines in the red blood cells of pancreatic cancer patients, as well as colorectal cancer patients, is higher than the reference value, which is the detection value of the biological sample from a healthy adult (Cancer. 1980 Jan. 1; 45(1):108-11). Ishikawa et al. also reported that in the oral tissues of healthy adults, putrescine wase 1048.98±626.75 ng/g and spermidine was 1260.77±883.12 ng/g, while in the oral tissues of oral cancer patients, putrescine was 7867.86±6073.54 ng/g and spermidine was 3573.15±3718.40 ng/g, also indicating the detection value of polyamines in the oral tissues of oral cancer patients is higher than the reference value, which is the detection value of the biological sample from a healthy adult (Sci Rep. 2016 Aug. 19: 6:31520). In addition, there are also relevant research reports on various cancers such as liver cancer, prostate cancer, breast cancer, lymphoma, fibroma, lung cancer or gastric cancer.

To evaluate the detection method of polyamines can be used to measure the amounts of the polyamines in the sample, the following trials are carried out:

### Trial (A).

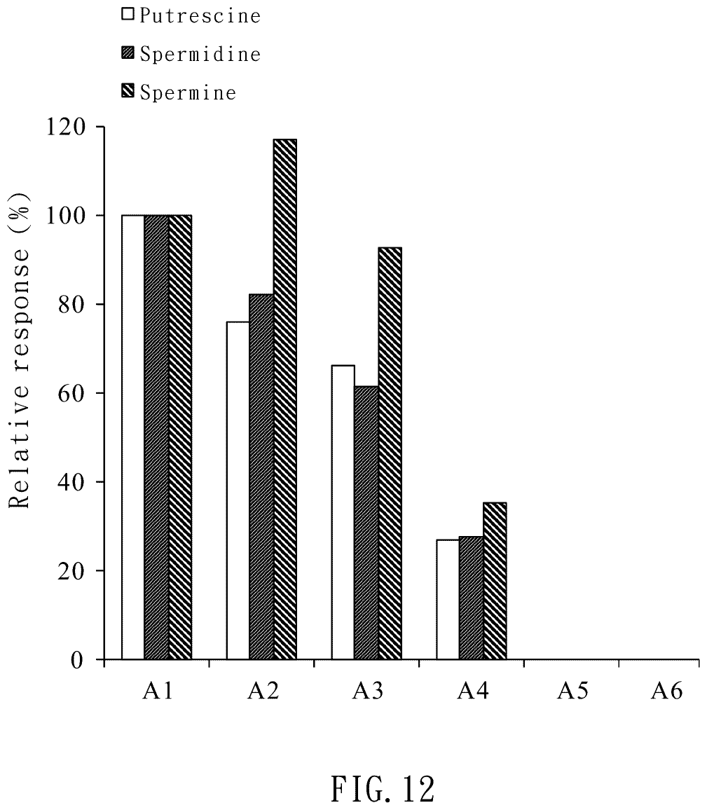

In trial (A), 100 μL of a polyamine solution (1 mM putrescine, 1 mM spermidine and 1 mM spermine, dissolved in water), 200 μL of a solution of derivatization reagent with a concentration of the derivatization reagent of 10 mM [dissolved in the working solution (ACN)] and 25 μL of a basic solution [10 mM of the base (NaOH), dissolved in water] are mixed uniformly to form the mixture. The mixture is heated by microwave (300 watts, 5 minutes), and then is placed in an ice bath to terminate the thiocarbamoylation reaction, obtaining the derivatization solutions of groups A1 to A6. Then, each of the derivatization solutions (1 μL) is analyzed by thin layer chromatography (TLC) under a wavelength of 254 nm. The formation of the thiourea derivative and the amounts of the formed thiourea derivative are shown in FIG. 12.

The derivatization reagents used in Trial (A) are listed in TABLE 2. The derivatization reagents of groups A5 and A6 are not the isothiocyanate with the isothiocyanate group.

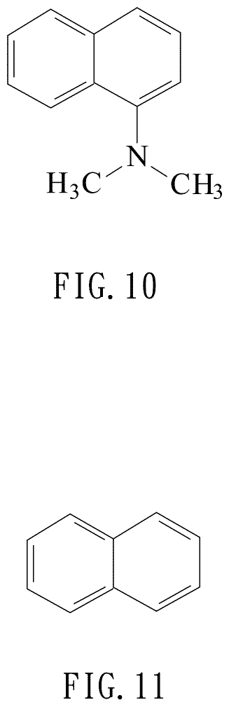

TABLE 2

| | | Chemical |
|---|---|---|
| Groups | Derivatization reagent | structure |
| A1 | 4-Dimethylamino-naphthylisothiocyanate (DNITC) | FIG. 3 |
| A2 | 1-Naphthyl isothiocyanate (NITC) | FIG. 4 |
| A3 | Benzyl isothiocyanate (BITC) | FIG. 5 |
| A4 | Ally isothiocyanate (AITC) | FIG. 6 |
| A5 | N,N-Dimethyl-1-naphthylamine (DNA) | FIG. 10 |
| A6 | Naphthalene | FIG. 11 |

Referring to FIG. 12, all of the derivatization solutions of groups A1 to A4 obtained from the derivatization reagents having the isothiocyanate groups contain the thiourea derivative, among which the derivatization reagent (DNITC) of group A1 shows the best effect. It is worthy to note that by comparing the derivatization reagents of groups A1 and A5, we can know that the thiocarbamoylation reaction is indeed carried out through the isothiocyanate group of the derivatization reagent. The same conclusion can also be made by comparing the derivatization reagents of groups A2 and A6.

### Trial (B).

In trial (B), 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution with a concentration of the base of 10 mM (dissolved in water) are mixed uniformly to form the mixture. After heating the mixture by microwave (300 watts, 5 minutes), the mixture is placed in the ice bath to terminate the thiocarbamoylation reaction, obtaining the derivatization solutions of groups B0 to B5. Then, 500 μL of the removing reagent (octane) and a powder mixture including the proton donor (citric acid; 25 mg) and the effervescent salt (KHCO₃; 10 mg) are added to the derivatization solutions of groups B0 to B5, respectively, and the derivatization solutions of groups B0 to B5 are placed at a temperature of −20° C. for a time period of 3 minutes, forming the middle-layer solutions of groups B0 to B5, which are used as the analytic solutions of groups B0 to B5, respectively. Finally, 8 μL of the analytic solutions of groups B0 to B5, respectively, are analyzed by narrow-bore HPLC-UV.

The bases used in Trial (B) are listed in TABLE 3. Group B0 indicates the control group without the base.

TABLE 3

| Groups | Base |
|---|---|
| B0 | None |
| B1 | Sodium hydroxide (NaOH) |
| B2 | Sodium bicarbonate (NaHCO₃) |
| B3 | Sodium carbonate (Na₂CO₃) |
| B4 | 4-Dimethylaminopyridine (DMAP) |
| B5 | Triethylamine (TEA) |

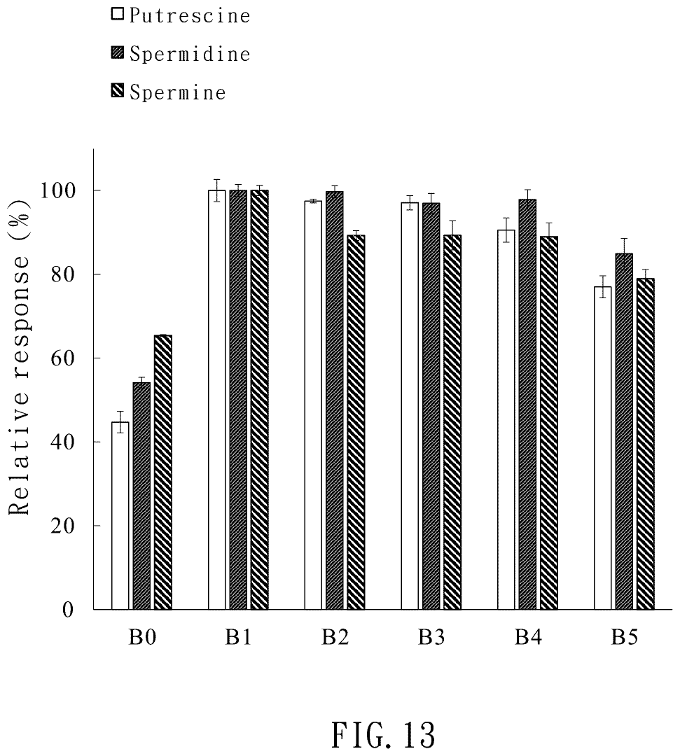

Referring to FIG. 13, adding the base can improve the progress of the thiocarbamoylation reaction, thus forming more thiourea derivatives (groups B1 to B5), among which sodium hydroxide (NaOH) of group B1 shows the best effect.

### Trial (C).

In trial (C), 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave, the mixture is placed in the ice bath to terminate the thiocarbamoylation reaction, obtaining the derivatization solutions of groups C1 to C4. Then, 500 μL of the removing reagent (octane) and the powder mixture including the proton donor (citric acid; 25 mg) and the effervescent salt (KHCO₃; 10 mg) are added to the derivatization solutions of groups C1 to C4, respectively, and the derivatization solutions of groups C1 to C4 are placed at a temperature of −20° C. for a time period of 3 minutes, forming the middle-layer solutions of groups C1 to C4, which are used as the analytic solutions of groups C1 to C4, respectively. Finally, 8 μL of the analytic solutions of groups C1 to C4, respectively, are analyzed by narrow-bore HPLC-UV.

The heating means used in Trial (C) are listed in TABLE 4. Group C0 is heated by means of a dry bath of 30° C., and groups C1 to C4 are heated by means of microwave at a power of from 100 watts to 750 watts.

TABLE 4

| Groups | Heating means |
|---|---|
| C0 | 30° C. dry bath |
| C1 | Microwave (100 W, 5 minutes) |
| C2 | Microwave (300 W, 5 minutes) |
| C3 | Microwave (550 W, 5 minutes) |
| C4 | Microwave (750 W, 5 minutes) |

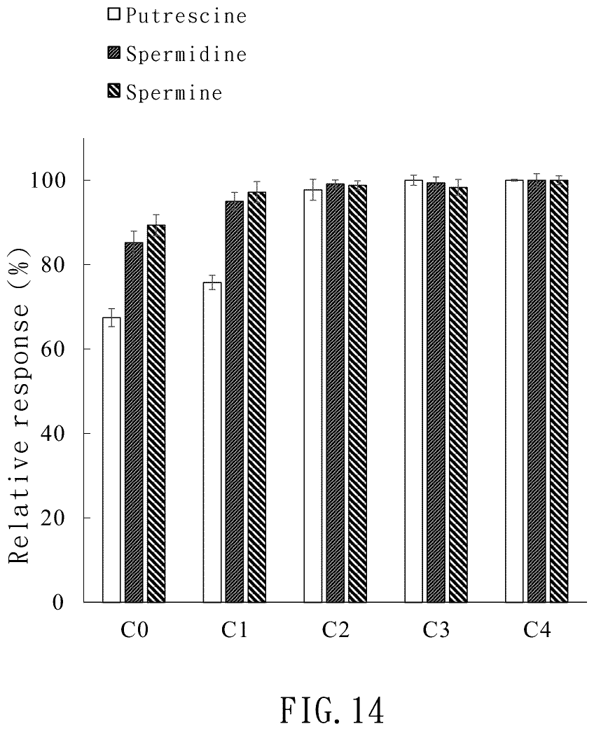

Referring to FIG. 14, heating the mixture by microwave can improve the progress of the thiocarbamoylation reaction, thus forming more thiourea derivatives (groups C1 to C4), among which 300 watts of group C2 shows the best effect.

### Trial (D).

In trial (D), 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solutions of groups D0 to D4. Then, 500 μL of the removing reagent and the powder mixture including the proton donor (citric acid; 25 mg) and the effervescent salt (KHCO₃; 10 mg) are added to the derivatization solutions of groups D0 to D4, respectively, and the derivatization solutions of groups D0 to D4 are placed at a temperature of −20° C. for a time period of 3 minutes, forming the middle-layer solutions of groups D0 to D4, which are used as the analytic solutions of groups D0 to D4, respectively. Finally, 8 μL of the analytic solutions of groups D0 to D4, respectively, are analyzed by narrow-bore HPLC-UV.

The removing reagents used in trial (D) are listed in TABLE 5. Group D0 indicants the control group without the removing reagent.

TABLE 5

| Groups | Removing reagent |
|---|---|
| D0 | None |
| D1 | Hexane |
| D2 | Octane |
| D3 | Decane |
| D4 | Dodecane |

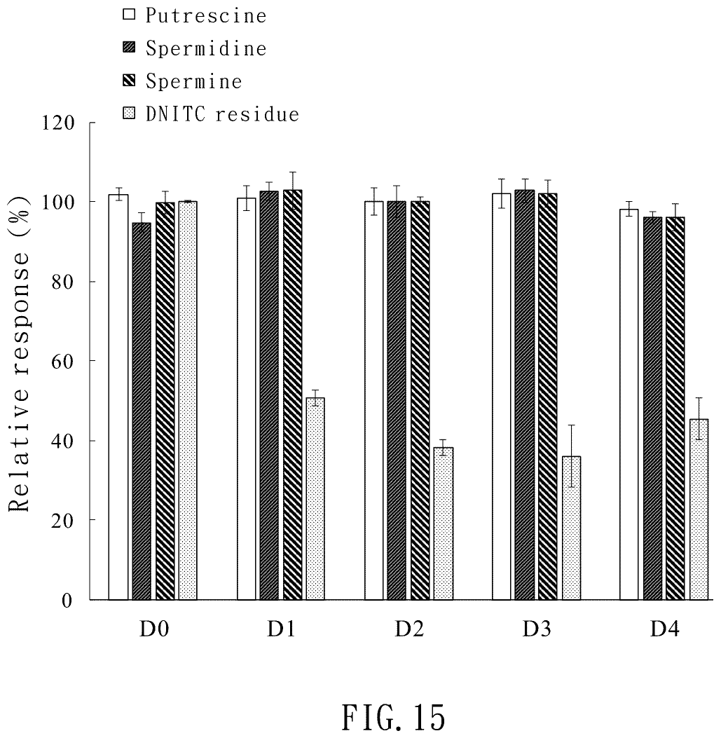

Referring to FIG. 15, adding the removing reagent to the derivatization solution can help to remove the excessive derivatization reagents (DNITC residue) (groups D1 to D4), among which octane of group D2 shows the best effect.

### Trial (E).

In trial (E), 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solutions of groups E0 to E3.

Then, 500 μL of the removing reagent (octane) and the powder mixture including the proton donor (citric acid; 25 mg) and the effervescent salt (KHCO₃; 10 mg) are added to the derivatization solution of group E1, and the derivatization solution of group E1 is placed at a temperature of −20° C. for a time period of 3 minutes, forming the middle-layer solution of group E1, which is used as the analytic solution of group E1. Finally, 8 μL of the analytic solution of group E1 is analyzed by narrow-bore HPLC-UV.

Moreover, 500 μL of the removing reagent (octane) is added to the derivatization solution of group E2, forming the upper-layer solution and the lower-layer solution. After removing the upper-layer solution, 500 μL of the removing reagent (octane) and the powder mixture including the proton donor (citric acid; 25 mg) and the effervescent salt (KHCO₃; 10 mg) are added to the lower-layer solution, and the lower-layer solution is placed at a temperature of −20° C. for a time period of 3 minutes, forming the middle-layer solution of group E2, which is used as the analytic solution of group E2. Finally, 8 μL of the analytic solution of group E2 is analyzed by narrow-bore HPLC-UV.

Furthermore, 500 μL of the removing reagent (octane) is added to the derivatization solution of group E3, forming the upper-layer solution and the lower-layer solution. After removing the upper-layer solution, 500 μL of the removing reagent (octane) is added to the lower-layer solution, forming the another upper-layer solution and the another lower-layer solution. After removing the another upper-layer solution, 500 μL of the removing reagent (octane) and the powder mixture including the proton donor (citric acid; 25 mg) and the effervescent salt (KHCO₃; 10 mg) are added to the another lower-layer solution, and the another lower-layer solution is placed at a temperature of −20° C. for a time period of 3 minutes, forming the middle-layer solution of group E3, which is used as the analytic solution of group E3. Finally, 8 μL of the analytic solution of group E3 is analyzed by narrow-bore HPLC-UV.

Moreover, group E0 indicates the control group without the removing reagent.

TABLE 6

| Groups | Number of removing step performed |
|---|---|
| E0 | 0 |
| E1 | 1 |
| E2 | 2 |
| E3 | 3 |

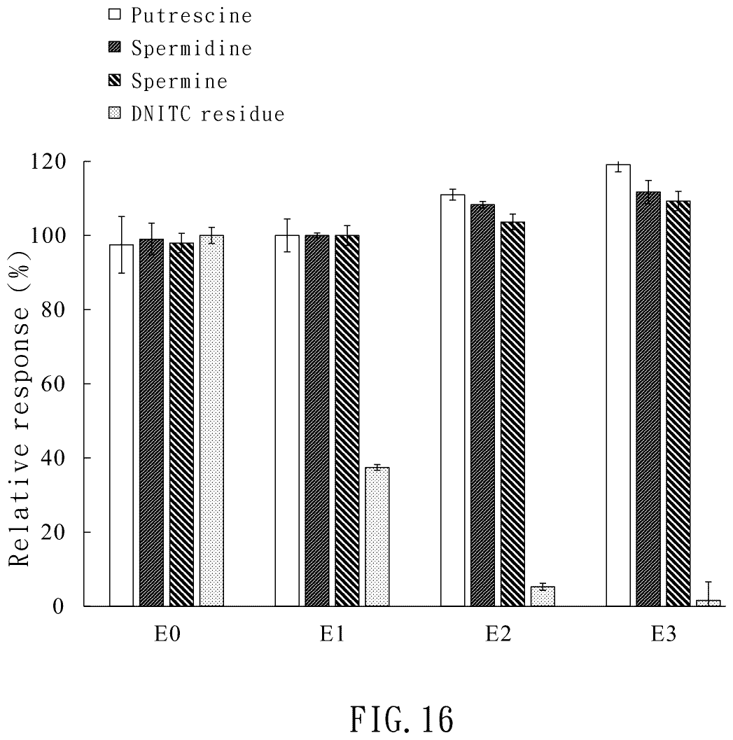

Referring to FIG. 16, performing multiple removing steps can help to remove the excessive derivatization reagents (DNITC residue) (groups E2 to E3).

### Trial (F).

In trial (F), 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solutions of groups F1 to F4. Then, 500 μL of the removing reagent (octane) and the powder mixture including the proton donor (shown in TABLE 7; 25 mg) and the effervescent salt (KHCO₃; 10 mg) are added to the derivatization solutions of groups F1 to F4, respectively, and the derivatization solutions of groups F1 to F4 are placed at a temperature of −20° C. for a time period of 3 minutes, forming the middle-layer solutions of groups F1 to F4, which are used as the analytic solutions of groups F1 to F4, respectively. Finally, 8 μL of the analytic solutions of groups F1 to F4, respectively, are analyzed by narrow-bore HPLC-UV.

TABLE 7

| Groups | Proton donor |
|---|---|
| F1 | Citric acid |
| F2 | Ammonium chloride (NH₄Cl) |
| F3 | Ammonium sulfate ((NH₄)₂SO₄) |
| F4 | Sodium dihydrogen phosphate (NaH₂PO₄) |

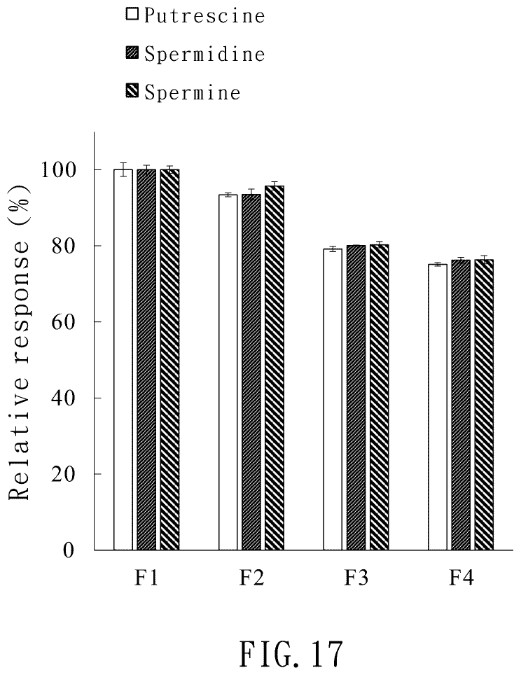

Referring to FIG. 17, by the use of the proton donors can help to separate the thiourea derivative (group F1 to F4), among which citric acid of group F1 shows the best effect.

### Trial (G).

In trial (G), 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solutions of groups G1 to G5. Then, 500 μL of the removing reagent (octane) and the powder mixture including the proton donor (citric acid; 25 mg) and the effervescent salt (shown in TABLE 8; 10 mg) are added to the derivatization solutions of groups G1 to G5, respectively, and the derivatization solutions of groups G1 to G5 are placed at a temperature of −20° C. for a time period of 3 minutes, forming the middle-layer solutions of groups G1 to G5, which are used as the analytic solutions of groups G1 to G5, respectively. Finally, 8 μL of the analytic solutions of groups G1 to G5, respectively, are analyzed by narrow-bore HPLC-UV.

TABLE 8

| Groups | Effervescent salt |
|---|---|
| G1 | Potassium bicarbonate (KHCO₃) |
| G2 | Sodium bicarbonate (NaHCO₃) |
| G3 | Ammonium bicarbonate (NH₄HCO₃) |
| G4 | Potassium carbonate (K₂CO₃) |
| G5 | Sodium carbonate (Na₂CO₃) |

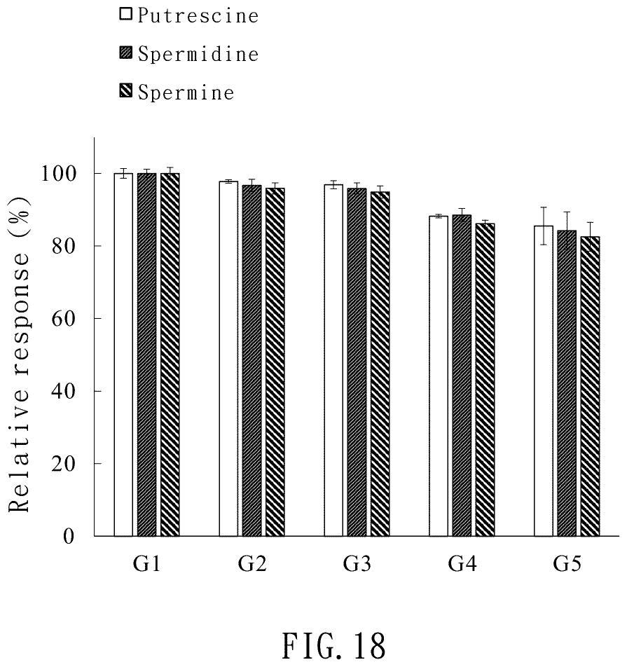

Referring to FIG. 18, by the use of the effervescent salts can help to separate the thiourea derivative (group G1 to G5), among which potassium bicarbonate (KHCO₃) of group G1 shows the best effect.

### Trial (H).

In trial (H), 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solutions of groups H0 to H1.

Then, 500 μL of the removing reagent (octane) and the powder mixture including the proton donor (citric acid; 25 mg) and the effervescent salt (KHCO₃; 10 mg) are added to the derivatization solution of group H1, and the derivatization solution of group H1 is placed at a temperature of −20° C. for a time period of 3 minutes, forming the middle-layer solution of group H1, which is used as the analytic solution of group H1. Finally, 8 μL of the analytic solution of group H1 is analyzed by narrow-bore HPLC-UV.

Moreover, 500 μL of the removing reagent (octane) and the powder mixture including the proton donor (citric acid; 25 mg) and the effervescent salt (KHCO₃; 10 mg) are added to the derivatization solution of group H0, and the derivatization solution of group H0 is placed at room temperature for a time period of 3 minutes, forming the middle-layer solution of group H0, which are used as the analytic solution of group H0. Finally, 8 μL of the analytic solution of group H0 is analyzed by narrow-bore HPLC-UV.

TABLE 9

| Groups | Temperature |
|---|---|
| H0 | About 25° C. |
| H1 | −20° C. |

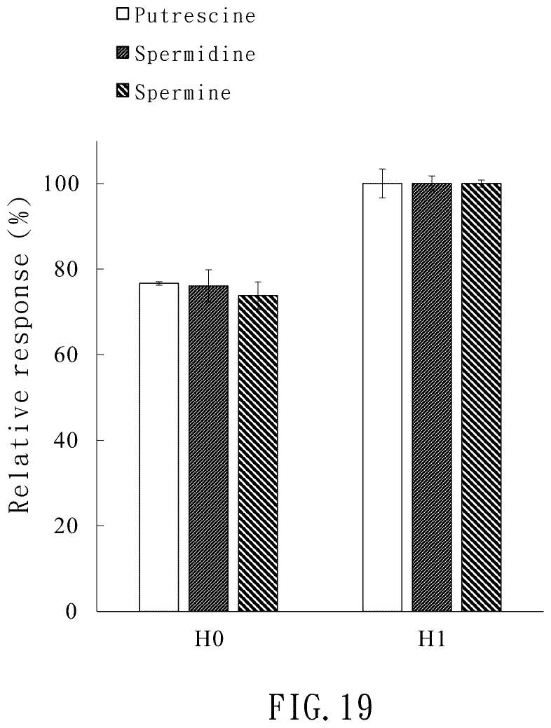

Referring to FIG. 19, the derivatization solution can be effectively layered by the cooling bath, thus the separation effect of the thiourea derivative can be improved (group H1).

### Trial (I).

The analytic solution of group I1: 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solution of group I1, which is used as the analytic solution of group I1.

The analytic solution of group I2: 100 μL of water (without putrescine, spermidine and spermine), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solution of group I2. Then, 500 μL of the removing reagent (octane) is added to the derivatization solution of group I2, forming the upper-layer solution and the lower-layer solution. After removing the upper-layer solution, 500 μL of the removing reagent (octane) is added to the lower-layer solution, forming the another upper-layer solution and the another lower-layer solution. After removing the another upper-layer solution, 500 μL of the removing reagent (octane) and the powder mixture including the proton donor (citric acid; 25 mg) and the effervescent salt (KHCO₃; 10 mg) are added to the another lower-layer solution, and the another lower-layer solution is placed at a temperature of −20° C. for a time period of 3 minutes, forming the middle-layer solution of group I2, which is used as the analytic solution of group I2.

The analytic solution of group I3: 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solution of group I3. Then, 500 μL of the removing reagent (octane) is added to the derivatization solution of group I3, forming the upper-layer solution and the lower-layer solution. After removing the upper-layer solution, 500 μL of the removing reagent (octane) is added to the lower-layer solution, forming the another upper-layer solution and the another lower-layer solution. After removing the another upper-layer solution, 500 μL of the removing reagent (octane) and the powder mixture including the proton donor (citric acid; 25 mg) and the effervescent salt (KHCO₃; 10 mg) are added to the another lower-layer solution, and the another lower-layer solution is placed at a temperature of −20° C. for a time period of 3 minutes, forming the middle-layer solution of group I3, which is used as the analytic solution of group I3.

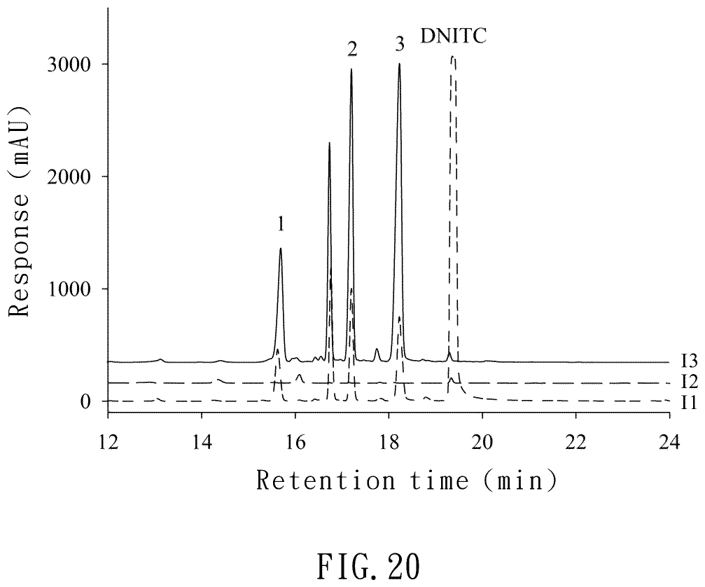

Finally, 8 μL of the analytic solutions of groups I1 to I3, respectively, are analyzed by narrow-bore HPLC-UV. The result is shown in FIG. 20, although the thiourea derivatives of polyamines (as shown in peaks 1, 2, and 3) are present in the analytic solution of group I1, the analytic solution of group I1 contains more derivatization reagent (DNITC), while in the analytic solution of group I3 after treatment, most of the derivatization reagent (DNITC) is removed, and there are still clear peaks representing the thiourea derivatives of polyamines.

### Trial (J).

In trial (J), 100 μL of the polyamine solution (200 μM putrescine, 200 μM spermidine and 200 μM spermine, dissolved in water), 200 μL of the solution of derivatization reagent [10 mM of the derivatization reagent (DNITC), dissolved in the working solution (ACN)] and 25 μL of the basic solution [10 mM of the base (NaOH), dissolved in water] are mixed uniformly to form the mixture. After heating the mixture by microwave (300 watts, 5 minutes), the mixture is placed in the ice bath to terminate the thiocarbamoylation reaction, obtaining the derivatization solution. Then, 500 μL of the removing reagent (octane) and the powder mixture including the proton donor (citric acid; 25 mg) and the effervescent salt (KHCO₃; 10 mg) are added to the derivatization solution, and the derivatization solution is placed at a temperature of −20° C. for a time period of 3 minutes, forming the middle-layer solution.

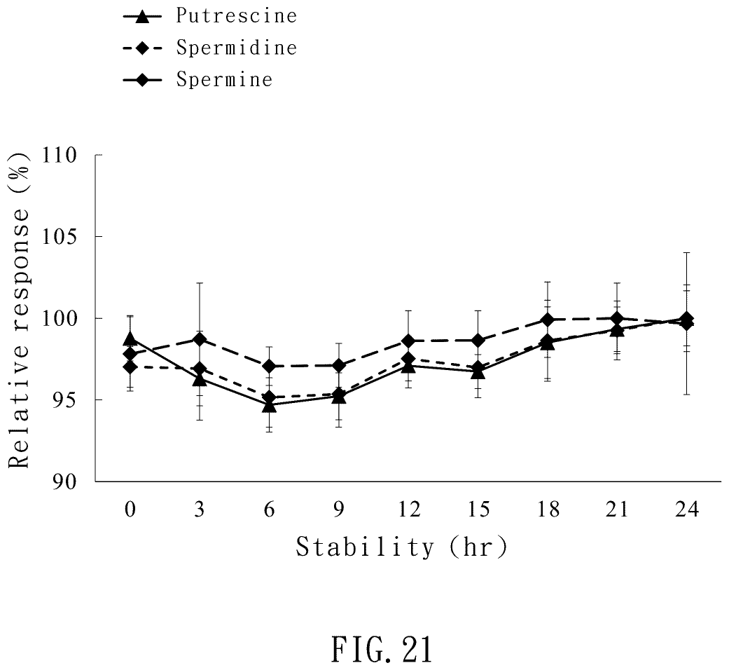

The middle-layer solution is stored in a temperature of 4° C. for subsequent use. 8 μL of the middle-layer solution is collected every 3 hours as the analytic solution, and then is analyzed by narrow-bore HPLC-UV. Comparing the changes in the area under a peak representing the thiourea derivatives of polyamines within 24 hours, as shown in FIG. 21, the concentration of the thiourea derivatives of polyamines does not change significantly, indicating that the formed thiourea derivative has good stability.

### Trial (K).

In trial (K), the polyamine solutions with a concentration of 1 μM, 5 μM, 25 μM, 100 μM and 200 μM are used, respectively. After obtaining the analytic solutions, the analytic solutions are analyzed by narrow-bore HPLC-UV. The linear regression equation and the coefficient of determination (R²) calculated by linear regression are listed in TABLE 10.

TABLE 10

| | | | Coefficient of |
|---|---|---|---|
| | | Linear regression | determination |
| | Sample | equation | (R²) |
| Intra- | Putrescine | y = 0.0058x+ 0.0060 | 0.9999 |
| batch | Spermidine | y = 0.114x + 0.0162 | 0.9999 |
| | Spermine | y = 0.0119x + 0.0023 | 0.9998 |
| Inter- | Putrescine | y = 0.0059x + 0.0058 | 0.9999 |
| batch | Spermidine | y = 0.0107x + 0.0190 | 0.9998 |
| | Spermine | y = 0.0121x − 0.0010 | 0.9999 |

Referring to TABLE 10, in either intra-batch or inter-batch analysis, the coefficient of determination (R²) is larger than 0.9998, indicating the detection method of polyamines has good liner correlation in the concentration range of 1 μM to 200 μM.

Then, the polyamine solutions with a concentration of 3 μM, 20 μM and 120 μM are used, respectively. After obtaining the analytic solutions, the analytic solutions are analyzed by narrow-bore HPLC-UV. The relative standard deviation (RSD) and the relative error (RE) calculated are listed in TABLE 11.

TABLE 11

| | Concentration | Intra-batch | | Inter-batch | |
|---|---|---|---|---|---|
| | (μM) | RSD (%) | RE (%) | RSD (%) | RE (%) |
| Putrescine | 3 | 2.96 | −1.80 | 3.84 | 1.21 |
| | 20 | 1.04 | 0.15 | 4.72 | −3.11 |
| | 120 | 0.53 | 0.53 | 1.13 | −0.91 |
| Spermidine | 3 | 5.41 | −1.21 | 2.30 | −6.82 |
| | 20 | 1.08 | 3.65 | 5.92 | 0.72 |
| | 120 | 2.01 | 3.92 | 3.23 | −0.42 |
| Spermine | 3 | 3.14 | −4.15 | 4.79 | 5.59 |
| | 20 | 1.84 | −2.20 | 4.58 | 1.39 |
| | 120 | 3.10 | 3.23 | 2.02 | −10.9 |

Referring to TABLE 11, in intra-batch analysis, the relative standard deviation (RSD) is smaller than 5.41%, and the relative error (RE) is smaller than −4.15%. Moreover, in inter-batch analysis, the relative standard deviation (RSD) is smaller than 5.92%, and the relative error (RE) is smaller than −6.82%. As a result, the detection method of polyamines has good accuracy and precision, that is, the detection method of polyamines shows good reliability.

### Trial (L).

Commercially available brown rice, wheat, buck wheat, oat, soybean and red wine are pre-treated such as grinding, protein precipitation, and then analyzed by the detection method of polyamines. The results are shown in TABLE 12.

TABLE 12

| | | Concentration | RSD |
|---|---|---|---|
| Food sample | Polyamines | (mg/kg) | (%) |
| Brown rice | Putrescine | N.D. | — |
| | Spermidine | 2.55 | 7.49 |
| | Spermine | 9.96 | 2.00 |
| Wheat | Putrescine | 6.40 | 2.26 |
| | Spermidine | 11.62 | 3.63 |
| | Spermine | 12.31 | 1.59 |
| Buck wheat | Putrescine | 4.07 | 2.08 |
| | Spermidine | 17.45 | 2.22 |
| | Spermine | 20.78 | 4.46 |
| Oat | Putrescine | 3.47 | 4.85 |
| | Spermidine | 6.43 | 0.62 |
| | Spermine | 4.01 | 3.12 |
| Soybean | Putrescine | 12.79 | 5.28 |
| | Spermidine | 83.95 | 1.85 |
| | Spermine | 24.35 | 3.82 |
| Red wine 1 | Putrescine | 1.22 | 17.29 |
| | Spermidine | 2.05 | 1.47 |
| | Spermine | 4.19 | 2.29 |
| Red wine 2 | Putrescine | 7.91 | 2.34 |
| | Spermidine | 4.27 | 0.72 |
| | Spermine | 5.91 | 7.65 |

Referring to TABLE 12, the detection method of polyamines can be applied to detect amounts of polyamines in the food sample.

### Trial (M).

Cancer cell lines including DU145 (prostate cancer cell line), T24 (bladder cancer cell line), Hep3B (liver cancer cell line), Huh7 (liver cancer cell line), HA22T (liver cancer cell line) and Mahlavu (liver cancer cell line) are used in trial (M). The cancer cells are lysed and analyzed by the detection method of polyamines. The results are shown in TABLE 13.

TABLE 13

| Cell line | | Concentration | RSD |
|---|---|---|---|
| sample | Polyamines | (ng/10⁷ cells) | (%) |
| DU145 | Putrescine | 1202.19 | 2.45 |
| | Spermidine | 1645.28 | 2.79 |
| | Spermine | 1436.08 | 3.89 |
| T24 | Putrescine | 542.30 | 9.53 |
| | Spermidine | 450.88 | 5.67 |
| | Spermine | N.D. | — |
| Hep3B | Putrescine | 2045.40 | 0.97 |
| | Spermidine | 1436.79 | 1.65 |
| | Spermine | 2881.44 | 5.36 |
| Huh7 | Putrescine | 3732.80 | 5.48 |
| | Spermidine | 630.65 | 7.61 |
| | Spermine | 2701.00 | 6.10 |
| HA22T | Putrescine | 3298.51 | 2.06 |
| | Spermidine | 787.20 | 3.07 |
| | Spermine | 2243.30 | 4.63 |
| Mahlavu | Putrescine | 373.00 | 0.36 |
| | Spermidine | 89.52 | 5.11 |
| | Spermine | 531.39 | 1.78 |

Referring to TABLE 13, the detection method of polyamines can be applied to detect amounts of polyamines in the cell line sample.

### Trial (N).

In trial (N), whole blood samples from 5 healthy volunteers are used. The whole blood samples are pre-treated such as protein precipitation, and then analyzed by the detection method of polyamines. The results are shown in TABLE 14.

TABLE 14

| Whole blood sample | Polyamines | Concentration (μM) |
|---|---|---|
| Volunteer 1 | Spermidine | 12.82 ± 0.44 |
| | Spermine | 2.16 ± 0.31 |
| Volunteer 2 | Spermidine | 12.52 ± 0.24 |
| | Spermine | 6.83 ± 0.53 |
| Volunteer 3 | Spermidine | 12.62 ± 0.14 |
| | Spermine | 5.74 ± 0.13 |
| Volunteer 4 | Spermidine | 10.56 ± 0.35 |
| | Spermine | 4.48 ± 0.47 |
| Volunteer 5 | Spermidine | 16.87 ± 0.15 |
| | Spermine | 6.31 ± 0.86 |

Referring to TABLE 14, the detection method of polyamines can be applied to detect amounts of polyamines in the whole blood sample.

Accordingly, by the thiocarbamoylation reaction between the amino group of the polyamines in the sample and the derivatization reagent (isothiocyanate with the isothiocyanate group), the thiourea derivative can be obtained. The thiourea derivative can be detected by various conventional methods. For example, after separation by liquid chromatography, gas chromatography, etc., the thiourea derivative can be detected using ultraviolet spectroscopy, fluorescence spectroscopy, or mass spectrometry. As such, the detection method of polyamines according to the present invention has great sensitivity, precision and accuracy, reducing sample usage.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A detection method of polyamines, comprising:
providing a sample with polyamines;
dissolving the sample and a derivatization reagent with an isothiocyanate group in a working solution to form a mixture;
performing a thiocarbamoylation reaction between the polyamines in the sample and the isothiocyanate group of the derivatization reagent to obtain a derivatization solution with a thiourea derivative; and
detecting the thiourea derivative to obtain a value of polyamines.

2. The detection method of polyamines as claimed in claim 1, wherein the derivatization reagent is 4-dimethylamino-naphthylisothiocyanate (DNITC), 1-naphthyl isothiocyanate (NITC), benzyl isothiocyanate (BITC) or ally isothiocyanate (AITC).

3. The detection method of polyamines as claimed in claim 1, wherein the working solution is acetonitrile (ACN), acetone, dimethyl sulfoxide (DMSO), pyridine, dimethylformamide (DMF), dimethylpropyleneurea (DMPU), sulfolane, tetrahydrofuran (THF), 1,2-dimethoxyethane (DME), 1,3-dioxolane (DOL), dimethyl carbonate (DMC) or water.

4. The detection method of polyamines as claimed in claim 1, further comprising: microwaving the mixture to drive the performance of the thiocarbamoylation reaction.

5. The detection method of polyamines as claimed in claim 4, wherein the mixture is microwaved at a power of from 100 watts to 750 watts.

6. The detection method of polyamines as claimed in claim 1, further comprising: adding a base to the mixture, followed by performing the thiocarbamoylation reaction in the mixture dissolved with the base.

7. The detection method of polyamines as claimed in claim 6, wherein the base is sodium hydroxide (NaOH), sodium bicarbonate (NaHCO₃), sodium carbonate (Na₂CO₃), 4-dimethylaminopyridine (DMAP) or triethylamine (TEA).

8. The detection method of polyamines as claimed in claim 1, further comprising: adding a removing reagent to the derivatization solution to remove fat-soluble interferents in the derivatization solution by the removing reagent and to form an upper-layer solution and a lower-layer solution, wherein the lower-layer solution contains the thiourea derivative, wherein the removing reagent is a solvent that is immiscible with the derivation solution and has a log P of from 3.5 to 7.0.

9. The detection method of polyamines as claimed in claim 8, wherein the removing reagent is hexane, octane, decane or dodecane.

10. The detection method of polyamines as claimed in claim 1, further comprising: adding a proton donor and an effervescent salt to the derivatization solution to produce carbon dioxide (CO_{2(g)}) by a neutralization reaction between the proton donor and the effervescent salt, and to form an upper-layer solution and a lower-layer solution, wherein the upper-layer solution contains the thiourea derivative.

11. The detection method of polyamines as claimed in claim 10, wherein the proton donor is citric acid, ammonium chloride (NH₄Cl), ammonium sulfate ((NH₄)₂SO₄) or sodium dihydrogen phosphate (NaH₂PO₄).

12. The detection method of polyamines as claimed in claim 10, wherein the effervescent salt is potassium bicarbonate (KHCO₃), sodium bicarbonate (NaHCO₃), ammonium bicarbonate (NH₄HCO₃), potassium carbonate (K₂CO₃) or sodium carbonate (Na₂CO₃).

13. The detection method of polyamines as claimed in claim 10, further comprising: after adding the proton donor and the effervescent salt to the derivatization solution, and before forming the upper-layer solution and the lower-layer solution, placing the derivatization solution in a cooling bath.

14. The detection method of polyamines as claimed in claim 1, further comprising: adding a removing reagent, a proton donor and an effervescent salt to the derivatization solution to remove fat-soluble interferents in the derivatization solution by the removing reagent, to produce carbon dioxide (CO_{2(g)}) by a neutralization reaction between the proton donor and the effervescent salt, and to form a upper-layer solution, a middle-layer solution and a lower-layer solution, wherein the middle-layer solution contains the thiourea derivative, and wherein the removing reagent is a solvent that is immiscible with the derivation solution and has a log P of from 3.5 to 7.0.

15. The detection method of polyamines as claimed in claim 14, wherein the removing reagent is hexane, octane, decane or dodecane.

16. The detection method of polyamines as claimed in claim 14, wherein the proton donor is citric acid, ammonium chloride (NH₄Cl), ammonium sulfate ((NH₄)₂SO₄) or sodium dihydrogen phosphate (NaH₂PO₄).

17. The detection method of polyamines as claimed in claim 14, wherein the effervescent salt is potassium bicarbonate (KHCO₃), sodium bicarbonate (NaHCO₃), ammonium bicarbonate (NH₄HCO₃), potassium carbonate (K₂CO₃) or sodium carbonate (Na₂CO₃).

18. The detection method of polyamines as claimed in claim 14, further comprising: after adding the removing reagent, the proton donor and the effervescent salt to the derivatization solution, and before forming the upper-layer solution, the middle-layer solution and lower-layer solution, placing the derivatization solution in a cooling bath.

19. A diagnostic method of cancers, comprising:
obtaining a biological sample from a suspected patient;
detecting polyamine level in the biological sample ex vivo by the detection method as claimed in claim 1 to obtain a detection value; and
comparing the detection value of the biological sample with a reference value, wherein the detection value is higher than the reference value indicates that the suspected patient suffers from cancer.

20. The diagnostic method of cancers as claimed in claim 19, wherein the cancer is liver cancer, prostate cancer, pancreatic cancer, colorectal cancer, breast cancer, lymphoma, fibroma, lung cancer, gastric cancer or oral cancer.
